(19) United States
(12) Patent Application Publication
(10) Pub. No.: US 2026/0294830 A1
(43) Pub. Date: Oct. 1, 2026

(54) AN INTELLIGENT CHARGE SWITCHABLE POLYMER-LIPID HYBRID SYSTEM FOR IN VIVO DELIVERY OF MOLECULES INCLUDING RNAs

(71) Applicant: QURCAN THERAPEUTICS INC., Toronto (CA)

(72) Inventors: Azhar Zahoor ABBASI, Milton (CA); Chunsheng HE, Woodbridge (CA); Mohammad Ali AMINI, Whitby (CA); Xiaoyu WU, Toronto (CA)

(21) Appl. No.: 19/489,826

(22) PCT Filed: Jun. 3, 2024
(86) PCT No.: PCT/CA2024/050740

§ 371 Date: Dec. 3, 2025

Related U.S. Application Data
(60) Provisional application No. 63/506,066, filed on Jun. 3, 2023.

(51) Int. Cl. A61K 9/51; A61K 31/7105; A61K 31/713; A61K 48/00; B82Y 5/00; B82Y 15/00; C12N 15/113; C12N 15/88

(52) U.S. Cl. CPC A61K 9/5123; A61K 9/5146; A61K 31/7105; A61K 31/713; A61K 48/0041; A61K 48/0091; B82Y 5/00; B82Y 15/00; C12N 15/113; C12N 15/88; C12N 2310/11; C12N 2310/141

(57) ABSTRACT

Among other things, in general, polymer-lipid nanoparticles (PLNPs) are disclosed that have charge switching properties which allow for efficient release of payloads in the pH environment of the endosome. PLNPs and methods of manufacture thereof are disclosed for charge switchable PLNPs having an anionic polymeric shell surrounding ionizable cationic lipids (also potentially including helper lipids and cholesterol) that are in complex with a cargo (such as, in a non-limiting example, therapeutically useful nucleic acids).

### CROSS-REFERENCE TO RELATED APPLICATION

This application claims the benefit of U.S. Provisional Patent Application Ser. No. 63/506,066, filed Jun. 3, 2023, which is hereby incorporated by reference in its entirety.

### BACKGROUND

Lipid nanoparticles (LNPs) are small carrier vehicles synthesized by using a variety of lipids. These carrier systems are able to load different molecules ranging from small drug molecules to large biomolecules. Furthermore, due to the amphiphilic nature of lipid used to develop these tiny carrier systems, LNPs provide the flexibility to load both hydrophilic and hydrophobic molecules and have been gaining much interest recently for therapeutic applications.

An mRNA vaccine induces a transient protein expression that is not only effective for infectious diseases, but also holds a potential to target and treat other diseases including cancer and rare genetic diseases. However, naked mRNA is inherently unstable and prone to rapid degradation by nucleases and self-hydrolysis.

Encapsulating the mRNA within LNPs protects the mRNA from extracellular ribonucleases and assists the intracellular delivery of these biomolecules at the proper site.

Once administered, LNPs need to protect mRNA from nuclease degradation in physiological fluids. Additionally, the LNPs should evade the rapid clearance by mononuclear phagocytic system (MPS) post systemic administration, and finally reach the target tissue, internalized by the target cells and escape the endosomes and release mRNA in cytoplasm of the cells to have higher transfection efficacy. To achieve an mRNA carrier system with such properties, LNPs are designed and developed using different excipient molecules to increase the loading of mRNA and improve the stability of LNP carrier system under physiological conditions. Generally, there are four main components of LNPs: 1) permanently cationic or ionizable cationic lipids, 2) helper lipids, 3) cholesterol, and 4) PEG-lipids. Different compositions of these components are used to tune the vehicle size to make an LNP suitable for injection.

The manufacturing of LNPs is based on the spontaneous self-assembly of nanoparticles due to electrostatic, hydrophobic and van der Waals interactions between different components and mRNA. Cationic or ionizable lipids are widely used for manufacturing, and are the major and most important component (constitute 30-60% of total lipids) of LNPs. The electrostatic interaction between positively charged lipids and negatively charged mRNA begins the formulation of lipid droplets. These droplets are further organized with other components to produce homogenous carrier vehicles. The other excipient components such as cholesterol, helper lipids and PEG-lipid also play a key role related to stability, clearance, and distribution of LNPs.

Recently, permanently cationic lipids have been replaced with pH-sensitive ionizable lipids due to concerns about tissue toxicity and lack of in vivo efficacy. Despite the benefits of PEG-lipid to improve the stability and properties of LNPs, its presence in LNPs results in immune response in human. Antibodies to PEG are thought not to be ordinarily common in human due to lower exposure of environmental PEG. However, recent studies have shown that COVID-19 mRNA vaccine can significantly induce or boost anti-PEG antibodies in humans. This could be one of the possible reasons that trigger the hypersensitivity reactions observed in humans exposed to PEGylated nanomedicines. Additionally, PEG antibodies can promote immune-mediated clearance of systemically delivered PEGylated nanomedicines and limit their efficacy.

Therefore, even though there has been significant development of LNPs during recent years and remarkable success of LNP-based COVID-19 vaccine, there is still room for further improvements in the design and development of more stable LNPs with improved surface chemistry that is biocompatible, safe and suitable for administration in human.

### SUMMARY

In accord with the invention, there is provided a method of synthesizing charge switchable polymer-lipid nanoparticles (PLNPs) encapsulating a cargo, comprising the steps of: solubilizing lipids in an alcohol or organic solvent; solubilizing the cargo in an acidic aqueous solvent; solubilizing an anionic polymer in an aqueous solvent; rapidly mixing the solubilized lipids and cargo under stirring to obtain a lipid/cargo complex; adding the solubilized anionic polymer aqueous solution to the lipid/cargo complex to form a polymer/lipid-cargo emulsion; and homogenizing the polymer/lipid-cargo emulsion to obtain cargo loaded PLNPs.

In an aspect of this invention, the N/P ratio (defined herein as the stoichiometry between protonatable nitrogen in the lipid and anionic phosphate in a nucleic acid cargo) is in the 2:1 to 8:1 range and the cargo is a nucleotide. In another aspect of this invention, the N/P ratio is around 4:1. In another aspect of this invention, the ratio of ionizable cation lipid:cholesterol:helper lipid is in the range of 30:30:40 to 70:20:10. In another aspect, the ratio of ionizable cation lipid:cholesterol:helper lipid is in the range of 40:40:20 to 60:30:10. In still another aspect, the ratio of ionizable cation lipid:cholesterol:helper lipid is around 50:30:20. In another aspect, the resulting PNLPs are between about 100 nm and about 200 nm in diameter. In still another aspect, the resulting PNLPs are less than about 500 nm in diameter. In still another aspect, the cargo is nucleotide-based. In still another aspect, the cargo is mRNA. In another aspect, the cargo is selected from the group consisting of siRNA, mRNA, miRNA, tRNA, RNAi, antisense oligonucleotides and DNA. In another aspect, the weight ratio of anionic polymer to total lipid is between 0.01:1 to around 6:1. In still another aspect, the weight ratio of anionic polymer to total lipid is around 3:1. In yet another aspect, the weight ratio of anionic polymer to total lipid is between 1.01:1 to 10:1. In another aspect of the invention, the anionic polymer is an amphiphilic polymer with several carboxylic groups on its polymer back bone and on its side chains. In still another aspect, the anionic polymer is made from one or more of polysaccharide, chitosan, chitosan derivatives, polyacrylic acid, poly(methyl methacrylate/methacrylic acid), poly(butadiene/maleic acid), poly(ethylene-alt-maleic anhydride), poly(meth acrylamide). In yet another aspect, the cationic ionizable lipids are ionizable fatty acid or phospholipid lipids with pKa below pH 7.0. In still another aspect, the phospholipids are selected from the group consisting of the ionizable phospholipids Dlin-MC3-DMA, Lipid H (SM-102), Dlin-KC2-DMA, DlinDMA, YSK13-C3, L319, 304O13, CKK-E12, 503O13, C12-200, Lipid 10, ALC-0315, C14-4, 306i10. In yet another aspect, the anionic polymer comprises a graft terpolymer comprising a polysorbate, maltodextrin, and one or more of MAA, C₁-C₂₅ acrylate, ethyl acrylate, C₁-C₂₅—N,N′-disubstituted amino-(C₁-C₂₅)-acrylate, 2-(dimethylamino)ethyl methacrylate, 2-(diisopropylamino)ethyl methacrylate, C₁-C₂₅ methacrylate stearyl methacrylate, methacryl-(C₁-C₂₅)—OH methacryl-(C₁-C₂₅)—NH₂, 2-methacryloyloxyethyl phosphorylcholine, DSPE-PEG-AC, and DSPE-PEG-ACA; and the lipid comprises DLin-MC3-DMA, cholesterol, and one or more of DSPC and DPPG.

In accord with the invention, there is provided a charge switchable polymer-lipid nanoparticle (PLNP) comprising: an anionic polymer, ionizable cationic lipids, cholesterol, a helper lipid, a cargo, and where the ionizable cationic lipids form a complex with the cargo, and the anionic polymer forms a shell surrounding the complex, and the shell of the PLNP presents a negative charge under physiological conditions and protonates when the pH <6.

In one aspect of the invention, the N/P ratio is in the 2:1 to 8:1 range and the cargo is a nucleotide. In another aspect, the N/P ratio is around 4:1 In still another aspect, the ratio of ionizable cation lipid:cholesterol:helper lipid is in the range of 30:30:40 to 70:20:10. In yet another aspect, the ratio of ionizable cation lipid:cholesterol:helper lipid is in the range of 40:40:20 to 60:30:10. In another aspect, the ratio of ionizable cation lipid:cholesterol:helper lipid is around 50:30:20. In still another aspect, the PNLPs are between about 100 nm and about 200 nm in diameter. In another aspect, the PNLPs are less than about 500 nm in diameter. In still another aspect, the cargo is mRNA. In still another aspect, the cargo is selected from the group consisting of siRNA, mRNA, miRNA, tRNA, RNAi, antisense oligonucleotides and DNA. In another aspect, the weight ratio of anionic polymer to total lipid is between 1.01:1 to 6:1. In still another aspect, the weight ratio of anionic polymer to total lipid is around 3:1. In another aspect, the weight ratio of anionic polymer to total lipid is between 1.01:1 to 10:1. In still another aspect, the anionic polymer is an amphiphilic polymer with several carboxylic groups on its polymer back bone and on its side chains. In another aspect, the anionic polymer is made from one or more of polysaccharide, chitosan, chitosan derivatives, polyacrylic acid, poly(methyl methacrylate/methacrylic acid), and poly(butadiene/maleic acid), poly(ethylene-alt-maleic anhydride), poly(meth acrylamide). In still another aspect, the cationic ionizable lipids are ionizable fatty acid or phospholipids lipids with pKa below pH 7.0. In still another aspect, the phospholipids are selected from the group consisting of the ionizable phospholipids Dlin-MC3-DMA, Lipid H (SM-102), Dlin-KC2-DMA, DlinDMA, YSK13-C3, L319, 304O13, CKK-E12, 503O13, C12-200, Lipid 10, ALC-0315, C14-4, 306i10. In yet another aspect, the anionic polymer comprises a graft terpolymer comprising a polysorbate, maltodextrin, and one or more of MAA, C₁-C₂₅ acrylate, ethyl acrylate, C₁-C₂₅—N,N′-disubstituted amino-(C₁-C₂₅)-acrylate, 2-(dimethylamino)ethyl methacrylate, 2-(diisopropylamino)ethyl methacrylate, C₁-C₂₅ methacrylate stearyl methacrylate, methacryl-(C₁-C₂₅)—OH methacryl-(C₁-C₂₅)—NH₂, 2-methacryloyloxyethyl phosphorylcholine, DSPE-PEG-AC, and DSPE-PEG-ACA; and the lipid comprises DLin-MC3-DMA, cholesterol, and one or more of DSPC and DPPG.

In accord with the invention, there is provided a method of synthesizing charge switchable polymer-lipid nanoparticles (PLNPs) encapsulating a cargo, comprising the steps of: solubilizing lipids in an alcohol or organic solvent; solubilizing the cargo in an acidic aqueous solvent; solubilizing an anionic polymer in an aqueous solvent; rapidly mixing the lipids and cargo under stirring to obtain a lipid/cargo complex; adding the anionic polymer aqueous solution to the lipid/cargo complex to form a polymer/lipid-cargo emulsion; and homogenization of the polymer/lipid-cargo emulsion to obtain cargo loaded PLNPs, where the lipids are permanently cationic lipids. In an aspect of the invention the permanently cationic lipids are one or both of Dioleoyl-3-trimethylammonium propane (DOTAP), 1,2-di-O-octadecenyl-3-trimethylammonium propane (DOTMA).

In accord with the invention, there is provided a charge switchable polymer-lipid nanoparticle (PLNP) comprising: an anionic polymer, permanently cationic lipids, cholesterol, a helper lipid, a cargo, and where the permanently cationic lipids form a complex with the cargo, and the anionic polymer forms a shell surrounding the complex, and the shell of the PLNP presents a negative charge under physiological conditions and protonates when the pH <6.

### BRIEF DESCRIPTION OF THE DRAWINGS

In order to better understand various exemplary embodiments, reference is made to the accompanying drawings, wherein:

FIG. 1 shows a Schematic diagram depicting the process of manufacturing of PLNPs as a carrier system for RNA delivery.

FIG. 2 shows a schematic diagram of PLNP structure.

FIG. 3 shows a schematic diagram to demonstrate the charge switchable mechanism of PLNPs to facilitate the delivery of RNAs intracellularly in the cytoplasm.

FIG. 4 shows measured charge switching property of PLNPs.

FIG. 5: shows the size distribution of synthesized PLNPs loaded with mRNA measured using Malvern Nanosizer.

FIG. 6: shows in vitro EGFP transfection of Hela cells using EGFP-mRNA loaded PLNPs.

FIG. 7: shows in vitro EGFP transfection of MDA-MB-231 cells using EGFP-mRNA loaded PLNPs.

FIG. 8: shows in vivo luciferase transfection of mouse liver following intravenous (IV) treatment with FLuc-mRNA loaded PLNPs.

FIG. 9: shows in vivo luciferase transfection of mouse spleen and lymph nodes following intravenous (IV) treatment with FLuc-mRNA loaded PLNPs.

FIG. 10: shows in vivo (10A) and ex vivo (10B) brain delivery of mRNA following intravenous (IV) treatment with Cy5 labelled Fluc-mRNA loaded PLNPs.

### DETAILED DESCRIPTION

### Figure Legend

10 Solubilized total lipids
12 Solubilized cargo
14 Cargo/lipid complex
16 Anionic or amphiphilic polymer
18 Polymer-lipid-cargo complex
20 Lipid-cargo complex
21 Cargo, RNAs
22 Anionic or amphiphilic polymer
23 Active sites (e.g., carboxylic) of the anionic or amphiphilic polymer
24 Ionizable cationic lipid
26 Cholesterol
28 Helper lipid
29 Negative surface charge of the PLNP
30 Negative surface charge of the PLNP
31 Cell
32 Lipid membrane of the early endosome
33 Ionizable lipid of the PLNP within the early endosome
34 Lipid membrane of the late endosome
35 Lysosome
36,37,38 Animation of potential mechanism of endosomal escape of PLNP/cargo to the cytosol of the cell
39 Nucleus
1000 Transfected cells
1010 Transfected cells
1030 Transfected liver cells
1040 Transfected spleen and lymph cells
1050 Epi-fluorescence illustrating mRNA level in brain
1060 Epi-fluorescence illustrating mRNA level in brain tissue

The term “alkyl” as used herein means a straight or branched chain hydrocarbon containing from 1 to 25 carbon atoms, preferably from 1 to 10 carbon atoms, more preferably 1, 2, 3, 4, 5, or 6 carbons. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “amino” as used herein means a —NH₂ group.

The term “carboxy” as used herein means a —COOH group, which may be protected as an ester group: —COO-alkyl.

The term “hydroxy” as used herein means an —OH group.

The term “nucleic acid” is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA) in the form of a messenger RNA, anti-sense, plasmid DNA, parts of a plasmid DNA or genetic material derived from a virus. Anti-sense is a polynucleotide that interferes with the function of DNA and/or RNA. The term nucleic acids—refers to a string of at least two base-sugar-phosphate combinations. Natural nucleic acids have a phosphate backbone, artificial nucleic acids may contain other types of backbones, but contain the same bases. Nucleotides are the monomeric units of nucleic acid polymers. The term includes deoxyribonucleic acid (DNA) and ribonucleic acid (RNA). RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids.

The term “siRNA” means a small inhibitory ribonucleic acid. The siRNA are typically less than 30 nucleotides in length and can be single or double stranded. The ribonucleotides can be natural or artificial and can be chemically modified. Longer siRNAs can comprise cleavage sites that can be enzymatically or chemically cleaved to produce siRNAs having lengths less than 30 nucleotides, typically 21 to 23 nucleotides. siRNAs share sequence homology with corresponding target mRNAs. The sequence homology can be 100 percent or less but sufficient to result in sequence specific association between the siRNA and the targeted mRNA.

By Cₓ, we mean herein an acyclic straight or branched hydrocarbon of longest length x; thus, C₁-C₅ includes methyl, ethyl, propyl, butyl, pentyl, isopropyl, etc.

Herein are taught novel charge switchable polymer-lipid nanoparticles (PLNPs), methods of their synthesis and manufacture, and their use for in vivo delivery of, e.g., RNAs for enhanced efficacy.

Herein are taught the design and development of charge switchable PLNPs. The PLNPs described herein are negatively charged under physiological pH (generally, the pH in the blood circulatory system; physiological conditions are the conditions in the blood circulatory system), with better stability, low particle aggregation due to electrostatic repulsion of particle due to net surface charge, and better stability under physiological fluid condition such as in blood circulation. Charge switching is an important advantage of PLNPs described herein, which advantage will be further explained below and throughout.

Charge switchable PLNPs are constructed so as to have a net negative charge when solubilized in the neutral or slightly basic conditions found at physiological pH. However, these PLNPs switch their net charge to positive once at the intracellular level upon encountering the significant increase in proton concentration at endosomal pH. Without being held to any particular theory, it is believed that this pH change causes: (1) positive charge activation from the ionizable cationic lipid (in which the “ionizable lipid” becomes, at least in part, actually ionized and cationic/positive in net charge) and (2) protonation of carboxylic acid from the anionic or amphiphilic polymer, resulting in, at least in part, a polymer that is marketedly less ionized and more neutral in net charge.

For example, at physiological pH (in blood) the carboxylic acid of an anionic polymer is substantially deprotonated and negatively charged, whereas the charge from ionizable cationic lipid is neutral. This gives a net negative surface charge of PLNPs.

Once PLNPs are taken up by the cell and reach the endosomal compartment where pH shifts to acidic (i.e., below 7; specifically as to endosomes, below about 6.5 and becoming particularly relevant once pH reaches 5.5-5.0), the PLNP is in a new protic environment. As the local pH of the endosomal environment begins to approach or fall below the pKa of the ionizable lipid. The lipid turns positively charged. Further, at endosomal pH the carboxylic acid group from anionic polymer starts to protonate and neutralize. As a result, the overall PLNPs charge switches to positive and favors the endosomal escape and shuffle of PLNPs to the cytoplasm cell compartment due to endosome membrane destabilization because of the electrostatic interaction between positively charged PLNPs and negatively charged endosome lipid membrane. Overall, charge switching PLNPs as described herein provide an advantageous means of payload delivery into the cytosol of cells.

We herein disclose a method for the synthesis of pharmaceutically acceptable multifunctional and colloidally stable biocompatible charge switchable polymer lipid nanoparticles where a polymer is used that results in a negative shell under physiological conditions. One preferred embodiment synthesizes a charge switchable PLNP using a graft terpolymer of poly(methacrylic acid)-polysorbate 80-maltodextrin to form the shell and a combination of ionizable cationic lipid, cholesterol, and helper lipid as the total lipid portion of the cargo/lipid complex (e.g., ionizable cationic lipid [DLin-MC3-DMA], cholesterol, and helper lipid distearoylphosphatidylcholine [DSPC]). Notably, PLNPs can be synthesized using this method that, upon entering the cell, largely do not travel to the lysosome and can therefore enhance likelihood of payload delivery into the cell.

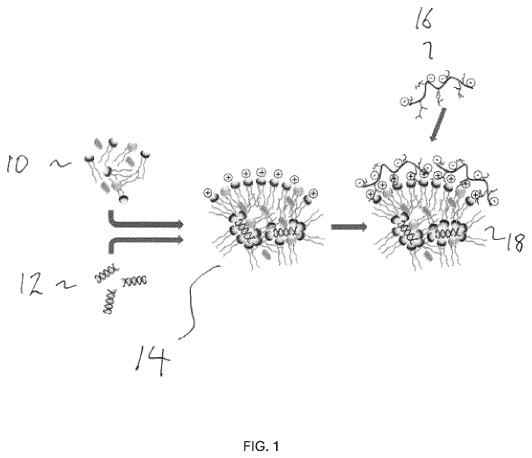

FIG. 1 shows a schematic diagram depicting the process of manufacturing of PLNPs as a carrier system for RNA delivery. The PLNPs are synthesized in part using the three-core material and methods that can be used for manufacturing of LNPs, utilizing a cationic lipid, cholesterol, and a helper lipid such as the DLin-MC3-DMA/cholesterol/DSPC given above. However, rather than use PEG-lipid as would be typical of LNPs, we herein teach that further adding an anionic polymer will improve the nanoparticle surface chemistry and stability and create the charge switching effect. Turning to FIG. 1, the nanocomplex synthesis starts with the rapid mixing of total lipids 10 in an alcohol or an organic solvent, preferably ethanol (a person skilled in the art will recognize that the choice of solvent will depend upon the specific lipid(s) used) and negatively charged RNA in aqueous solution 12 at a low pH below the pKa of ionizable lipid (preferably an acetate buffer [pH 4.0-5.0]) (a person skilled in the art will recognize that the pKa of the ionizable lipid, and thus the choice of buffer or other aqueous solvent, will depend upon the specific lipid[s] used). The hydrophilic head of cationic lipid interacts with the RNA, due to electrostatic interaction and the ionizable cationic lipids covers the RNA to form a lipid/RNA complex. Further change in the polarity of the solution of the lipids (including ionizable lipid, and cholesterol, and helper lipids, as will be known to a person skilled in the art; see for context a review on lipid composition in LNPs: Albertsen et al, Advanced Drug Delivery Reviews 2022 September; 188:114416, hereby incorporated by reference) creates a shell around multiple lipid-RNA complexes due to electrostatic, hydrophobic and Van der Waal interactions creating a positively charged large complex 14.

Continuing with FIG. 1, the manufacturing process proceeds with the addition of aqueous anionic polymer 16 which through further interactions with the droplets 14 (due, at least in part, to electrostatic and hydrophobic interactions), covers the surface of lipid-RNA complex 14 to form a polymer/lipid-RNA emulsion 18. The polymer-lipid-RNA complex 18 is then processed through sonication or homogenization or microfluidic technique to produce the small PLNPs.

In the process as illustrated in FIG. 1, the cargo carried by the PNLP is RNA. However, the synthesis described in the discussion of FIG. 1 can be used for any nucleic acid or nucleotide, including siRNA, mRNA, miRNA, RNA, tRNA, RNAi, antisense oligonucleotides and DNA (ideally with the lipids being selected to form a complete complex around the specific cargo).

As known to a person skilled in the art, further purification, buffer exchange to physiological pH and concentration of nanoparticles can be used as desired, as long as the PLNP particles retain a negative charge due to presence of anionic polymer on the surface.

In this synthesis, assuming the cargo is a nucleic acid or nucleotide, a preferred ratio of ionizable cation lipid:cholesterol:helper lipid is in the range of 40:40:20 to 60:30:10, although it can be broader, for example 30:30:40 to 70:20:10. A more preferred range is 50:30:20. Again assuming the cargo is a nucleic acid or nucleotide, the weight ratio of anionic polymer to total lipid can go as high as 10:1, but a preferred weight ratio of anionic polymer to total lipid is between 0.01:1 or 0.5:1 to 6:1, and more preferably is around 3:1.

The diameter of the PNLP resulting from this synthesis can be controlled at the homogenization and sonification stage, as known to persons skilled in the art. For the purposes of the PNLP being best designed to deliver a payload to the cytoplasm of a cell after delivery the bloodstream, a preferred diameter is 100 nm-200 nm. For EPR effects (Enhanced Permeation and Retention) the PNLPs should be less than 500 nm in diameter.

It is notable that, unlike typical lipid nanoparticles, the PLNPs produced by the above method do not have any exposed lipids on the shell or surface of the PLNP. As a result, the PNLPs produced by this synthesis have increased stability and reduce immunogenic reactions, since there are no exposed lipids for external interactions.

The cationic ionizable lipid 10 can be selected from a range of fatty acid, or different ionizable phospholipids (including but not limited to Dlin-MC3-DMA, Lipid H (SM-102), Dlin-KC2-DMA, DlinDMA, YSK13-C3, L319, 304O13, CKK-E12, 503O13, C12-200, Lipid 10, ALC-0315, C14-4, 306i10) or any ionizable lipid with pKa at or below pH 7.0. In some preferred embodiments, the cationic ionizable lipid has an apparent pKa between about 5.5 and about 7.0. In some preferred embodiments, the cationic ionizable lipid has an apparent pKa between about 6.0 and about 7.0.

The helper lipid can be any zwitterionic phospholipids, including but not limited to phosphatidylcholine, phosphatidylethanolamine, phosphatidylserine, and sphingomyelin such as DSPC, DPPC.

The anionic polymer (which preferably is amphiphilic) can be synthesized from the range of backbone material including polysaccharide, polysaccharides with amine groups (such as chitosan and chitosan derivatives), poly(acrylic acid), poly(methyl methacrylate/methacrylic acid), poly(butadiene/maleic acid), poly(ethylene-alt-maleic anhydride), poly(meth acrylamide). To be a preferred anionic polymer in accord with the invention, in some embodiments, the polymer features shorter glucose as, or on, the side chains (such as oligomeric maltodextrins or oligosaccharides or both) and hydroxyl-active groups on the backbone. The aforementioned materials are preferably selected to avoid pH-dependent swelling under physiological conditions.

The monomers of polymer in the sidechains of the polymer can be selected from range of materials including PS80, fatty acid with unsaturated chains (such as oleic acid), and phospholipids with unsaturated fatty chains, in all cases preferably with carboxylic groups.

In another embodiment of the synthesis described above, the PLNPs can be also obtained using permanently charged cationic phospholipids, including but not limited to Dioleoyl-3-trimethylammonium propane (DOTAP), 1,2-di-O-octadecenyl-3-trimethylammonium propane (DOTMA). In this situation, the polymer-lipid composition (referred to above as the weight ratio of polymer to lipid) needs to be adjusted by increasing the weight ratio of polymer to lipid to the point where the shell of the PNLP is negative under physiological conditions. The ratio depends upon the size of the cargo as well as the amount of permanently charged cationic lipid used to form a complex with the cargo; the amount of polymer must be sufficient to completely cover the complex(es) and so create a negatively charged shell.

The synthesis described above can be implemented using clinically approved ionizable cationic lipids, cholesterol and helper lipids that are used for clinically approved conventional LNPs. (A person skilled in the art should be aware that some of the materials described in the description of the synthesis above are not clinically approved.)

Importantly, in the present invention the fourth material typically used for construction of PEG-lipid LNPs, (such as 1,2-dimyristoyl-rac-glycero-3-methoxypolyethylene glycol-2000 [PEG2000-DMG] or other PEG-lipid conjugates) has been replaced by anionic/amphiphilic polymer to form the present PLNPs, further improving nanoparticle surface chemistry and stability over LNPs. Compared to conventional LNPs that have a net neutral charge under physiological condition, the present PLNPs described in this invention have net negative surface charge under physiological conditions (i.e., the condition in the blood) making them more stable during blood circulation.

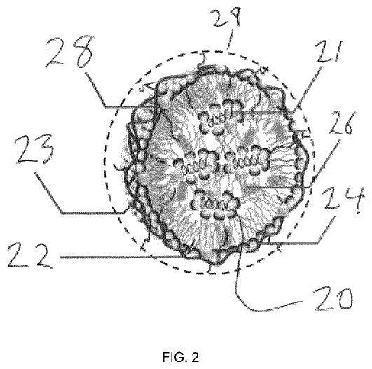

FIG. 2 shows a schematic diagram of the charge switching PLNP structure. Turning to FIG. 2, a charge switching PLNP is illustrated, including a coating of lipid-RNA complex 20 with anionic polymer 22 having carboxylic acid active sites 23. The polymer interacts with the lipid-RNA complex due to electrostatic and hydrophobic interactions. The PLNPs are synthesized as described above in reference to FIG. 1 using the three-core material used for manufacturing of LNPs such as a cationic lipid 24, cholesterol 26 and helper lipid 28. The fourth component introduced to produce the PLNPs is an anionic polymer 22 with carboxylic acid groups and pKa of ~pH in a preferred range of 5.0-6.0, although it is possible to use anionic polymer 22 with a lower pH range. Unlike LNPs that are neutral at physiological condition, the proposed system has negative surface charge 29 under physiological pH and has a better stability due to electrostatic repulsion between particles.

The net negative charge of these PLNPs reduces their aggregation due to electrostatic repulsion during storage, during preparation in suspension and following administration in blood circulation. Replacing PEG-lipids with biocompatible anionic polymers also helps to reduce hypersensitivity reaction and side effect related to PEG that are often observed in humans.

The novel PLNPs described in this invention also offer multiple chemically active surface groups 23 on the shell of the PLNP that can be functionalized with different cell or disease targeting molecules such as small molecules, peptides or proteins.

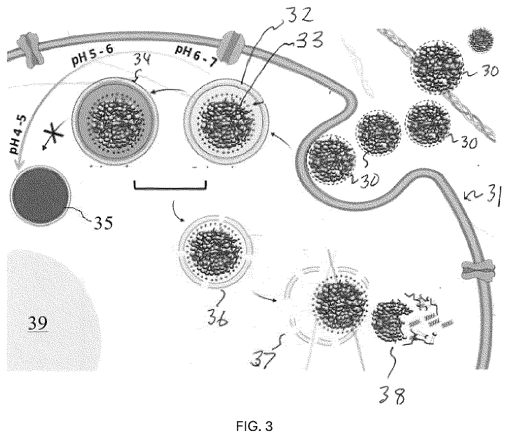

FIG. 3 shows a schematic diagram to demonstrate the charge switchable mechanism of PLNPs to facilitate the delivery of RNAs intracellularly in the cytoplasm. Turning to FIG. 3, the surface 30 of PLNPs is negatively charged during blood circulation improving the PLNP's stability under physiological condition without the use of PEG, due to electrostatic repulsion between particles. Once PLNPs are taken up by the cells 31 and reach the early endosomes 32 the pH is below 6.0, or below the pKa of ionizable lipids, and the ionizable lipid 33 begins to turn positive. However, at this low pH the polymer carboxylic acid groups on surface 30 start to protonate, and polymer charge starts to neutralize and continues protonating until the polymer charge turns positive. As a result, the net charge of PLNPs is taken up by the ionizable lipids 33. This switches the charge of PLNPs from negative to positive at pH <6.0 which is the case for endosomal pH (note that the pH bellow which this occurs depends upon the choice of lipid). This charge switching creates more interaction of PLNPs and endosomal 32 lipid membrane (illustrated as 34), causing a disruption in the membrane layer and finally escape of the PLNPs from endosome to cytoplasm (illustrated as 36, 37 and 38). In the cytoplasm the pH is higher and there is less interaction of RNA and ionizable lipid and the shell dissipates, which facilitate the release of RNA from the PLNPs to the cytoplasm.

It should be noted that, as seen in FIG. 3, in contrast to typical LNPs, PNLPs described herein do not travel to the lysosome.

In a preferred embodiment, the anionic polymer is graft terpolymer of poly(methacrylic acid)-polysorbate 80-maltodextrin, the ionizable cationic lipid is DLin-MC3-DMA, and the helper lipid is DSPC (Distearoyl-sn-glycero-3-phosphocholine). As an alternative, DPPG (dipalmitoyl-rac-glycero-3-phosphoglycerol) can also be used. This PNLP can be used with a cargo of RNA.

In a second preferred embodiment, the anionic polymer is graft terpolymer of poly(methacrylic acid)-oleic acid-maltodextrin, the lipid is ionizable cationic lipid DLin-MC3-DMA, and the helper lipid is DSPC (as an alternative, DPPG can also be used) and cholesterol. This PNLP can be used with a cargo of RNA.

In some embodiments, the anionic polymer is a graft terpolymer of poly(methacrylic acid)-polysorbate 80-maltodextrin. Briefly, polysorbate 80, maltodextrin, and methacrylic acid are combined in an emulsion polymerization in the presence of potassium persulfate (KPS), sodium thiosulfate (STS), and water at 70 degrees Celsius:

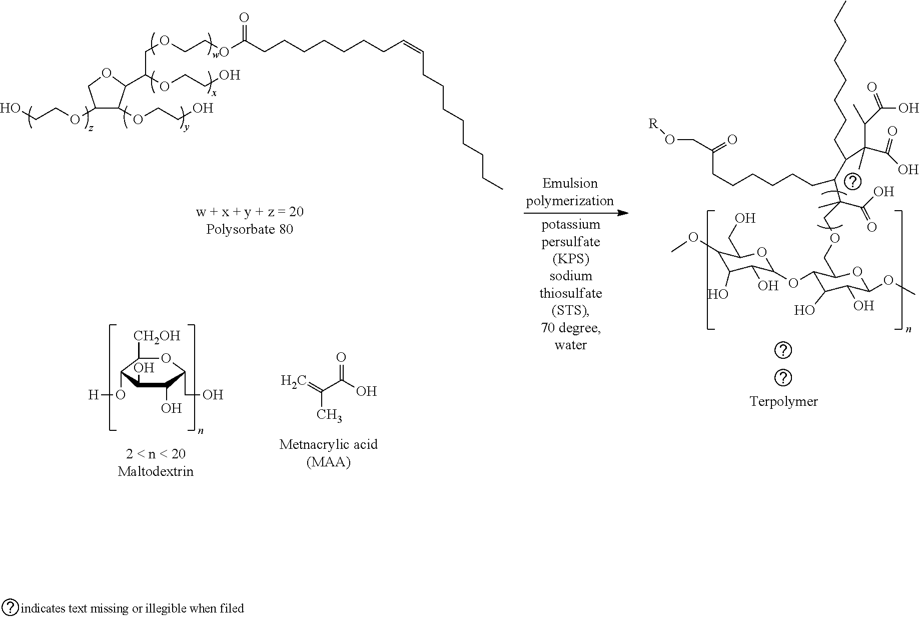

It should be noted that the illustrated resultant terpolymer above includes a backbone carboxylic acid group for each MAA unit in the polymer and the polysorbate includes a side chain carboxylic acid group; the effect of which is to provide several carboxylic acid groups per unit of terpolymer. In some embodiments, the quantity of maltodextrin or polysorbate can be varied in a manner understood by those skilled in the art to change the average number of terpolymer units so decorated. In some embodiments, a polysorbate with a different value of w, x, y, or z (or combinations thereof) can be used to vary the terpolymer structure.

In some embodiments, in lieu of MAA, a C₁-C₂₅ acrylate is used in the above synthesis. In some embodiments, ethyl acrylate is used. In some embodiments, a C₁-C₂₅-disubstituted amino C₁-C₂₅ acrylate is used (where Cₓ may be a different x in each case). In some embodiments, 2-(dimethylamino)ethyl methacrylate is used. In some embodiments, 2-(diisopropylamino)ethyl methacrylate is used. In some embodiments, a C₁-C₂₅ methacrylate is used. In some embodiments, stearyl methacrylate is used. In some embodiments, methacryl-(C₁-C₂₅)—OH is used. In some embodiments, methacryl-(C₁-C₂₅)—NH₂ (with hydrochloride as complex ion, as applicable) is used.

In some embodiments, in lieu of MAA, a phosphorylcholine is used. In some embodiments, 2-methacryloyloxyethyl phosphorylcholine is used. In some embodiments, DSPE-PEG-AC is used. In some embodiments, DSPE-PEG-ACA is used.

It is also possible to synthesize such a terpolymer by generally following the approach given in U.S. Pat. No. 10,233,277 (hereby incorporated by reference in its entirety).

Further details of the synthesis of graft terpolymers useful as anionic polymers for manufacture of PLNPs can be found in the below Example.

### EXAMPLES

### Chemicals and Reagents

Anionic polymer, a graft terpolymer of poly(methacrylic acid)-polysorbate 80-maltodextrin was synthesized as previously described herein. The terpolymer was made of polymethacrylic acid, and polysorbate 80 (PS80) grafted onto maltodextrin. For the polymer synthesis, the required amount of maltodextrin was dissolved in 150 mL of distilled water by heating at 70 degrees Celsius for 30 min. The solution was purged with N₂ to remove any dissolved oxygen. Subsequently, required amounts of PS80, KPS, and STS were added to the maltodextrin solution while being stirred. After 10 min, the reaction was started by addition of 1.55 g of nitrogen purged MAA. Opalescence appeared after 5 min and the reaction was continued for 4 h at 70° C. to ensure complete grafting. The product was washed multiple times using the tangential flow filtration (TFF) system (molecular weight cutoff 10 kDa) extensively against Milli-Q water. The purified polymer was then dried at 50 degrees Celsius for 24 h and stored in a desiccator for future use.

Ionizable cationic lipid DLin-MC3-DMA (4-(dimethylamino)-butanoic acid, (10Z,13Z)-1-(9Z,12Z)-9,12-octadecadien-1-yl-10,13-nonadecadien-1-yl ester; CAS number 1224606-06-7) was obtained from Cedarlane Canada, Cholesterol was obtained from Spectrum Chemical USA, DSPC was obtained from NOF America, and anhydrous ethanol was obtained from Greenfield Canada. EGFP-mRNA and FLuc-mRNA were obtained from Trilink biotech USA. All chemicals and reagents were used as received.

### Cell Line and Maintenance

The Human cervical cancer cells HeLa and human breast cancer cell line MDA-MB-231 were used for in vitro studies. Monolayers of cells were cultured on 75 cm2 polystyrene tissue culture flasks at 37° C. in 5% CO2/95% air humidified incubator. Cancer cells were maintained in α-minimal essential medium, supplemented with 10% fetal bovine serum. Cells grown to confluence were trypsinized with 0.05% trypsin-EDTA, diluted ( 1/10) in a fresh growth medium and reseeded.

### Experimental Animals

For in vivo transfection efficacy studies 8 weeks old female Balb/c mice (Jackson laboratory, Maine, USA) were used. The animals had free access to food and water throughout the study.

Preparation and Characterization of mRNA PLNPs

The manufacturing of PLNPs followed the method described above in association with FIG. 1, using an oil-in-water emulsion method and homogenization combined with sonification. Briefly, lipids were suspended in ethanol at a concentration of 6.6 mg/mL keeping the lipid ratio as described above. The lipid solution was rapidly mixed with mRNA in acetate buffer pH 4.5 (166.6 μg/mL) by keeping the N/P ratio (from 2:1 to 8:1) at 30° C. The aqueous buffer was 2-fold higher in volume to ethanol. After the pre-determined time of 3 minutes anionic polymer suspended in aqueous solution was added. The polarity change in the reaction mixture formed the oil-in-water emulsion and self-assembly of polymer-lipid particles. The emulsion is processed through sonication or homogenization to produced small PLNPs. The final PNLPs were transferred to ice cold RNAs free water. PLNPs were further purified, and buffer was exchanged with RNAs free water using the Amicon ultra centrifuge filter (100 kDa) at 2000-4500 rpm. Finally, PLNPs are passed through 0.22 μm syringe filter for sterilization. All aqueous solution were prepared using RNAs free water. Pipette tips and all instrument used during synthesis was cleaned with RNAseZap solvent. Finally, PLNPs were lyophilized using a freeze dryer at −80° C. in the presence of sucrose as a cryoprotectant.

### Determination of Particle Size by Dynamic Light Scattering

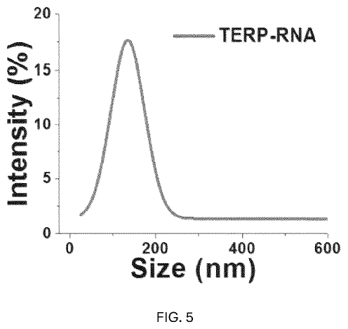

PLNPs size was measured by dynamic light scattering (DLS), using a Malvern Nano-ZS (Malvern, Worcestershire, UK) apparatus. The particle size was measured at 37° C. with a laser beam at a detection angle of 90°. The purified PLNPs were dispersed in distilled in water and particle size for each sample was measured three times and the average of the triplicate was reported. The intensity-weighted mean diameter was used as the hydrodynamic size since it is calculated directly from the original data and more reproducible than volume-weighted and number-weighted mean diameter. The particle size distribution was evaluated using polydispersity index (Pdl). Generally, particles with Pdl values smaller than 0.2 are considered monodisperse. FIG. 5 shows the size distribution of synthesized PLNPs loaded with mRNA measured using Malvern Nanosizer.

### Zeta Potential Measurement

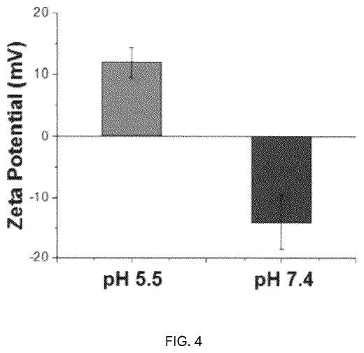

To study the effect of pH on surface charge, particles zeta potential was measured. The PLNPs suspension was diluted with buffer solutions of different pH and zeta potential was then measured using a Malvern zeta sizer Nano-ZS (Malvern, Worcestershire, UK). FIG. 4 shows charge switching property of PLNPs. Turning to FIG. 4, the zeta potential of the PLNPs was measured at pH 7.4 (physiological pH) and at pH 5.5 (endosomal pH).

In Vitro mRNA Transfection Using PLNPs

For in vitro transfection studies cells were seeded overnight in an eight-chambered glass bottomed dish at a density of 10,000 cells per well. Next day, cells were washed with serum-free DMEM, and EGFP mRNA loaded PLNPs were added at a final concentration of 150 ng mRNA per well. After mRNA-PLNPs treatment, cells were incubated for 6 h at 37° C. After required time, media was removed, and PBS solution was added. Cells were imaged using a Confocal laser scanning microscope with appropriate laser for GFP detection.

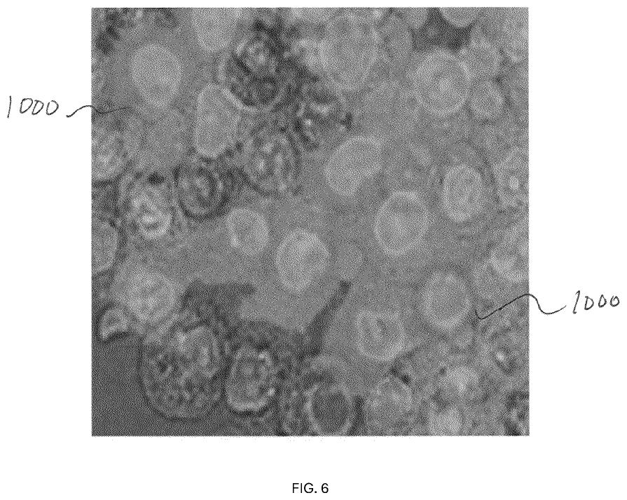

FIG. 6 shows in vitro EGFP transfection of Hela cells using EGFP-mRNA loaded PLNPs. The PLNPs were synthesized as described above using the commercially available EGFP-mRNA. Cells were treated for six hours before the imaging. Turning to FIG. 6, the transfected cells are labelled 1000 and are shown in green.

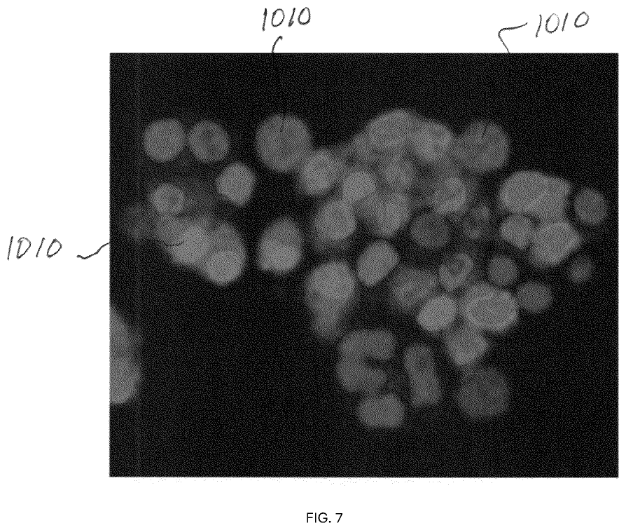

FIG. 7 shows in vitro EGFP transfection of MDA-MB-231 cells using EGFP-mRNA loaded PLNPs. The PLNPs were synthesized as described above using the commercially available EGFP-mRNA. Cells were treated for six hours before the imaging. Turning to FIG. 7, the transfected cells are labelled 1010 and are shown in green.

In Vivo mRNA Transfection Using PLNPs

For in vivo transfection experiment 8-weeks old female BALB/c mice were used. Briefly, animals were injected IV through the tail vain at a dose of 0.5-1.5 mg/kg of mRNA in 150 μL of 5% dextrose. Animals were left in cage for 6 hours. After 6 hours, expression of Fluc was analyzed by bioluminescence imaging (BLI) after intraperitoneal (I.P) injection of D-luciferin at 150 mg/kg. Animals were imaged within 5-10 min after D-Luciferase injection using a IVIS 200 system (Xenogen product line; Perkin-Elmer). During the in vivo imaging, animals were anesthetized with isoflurane.

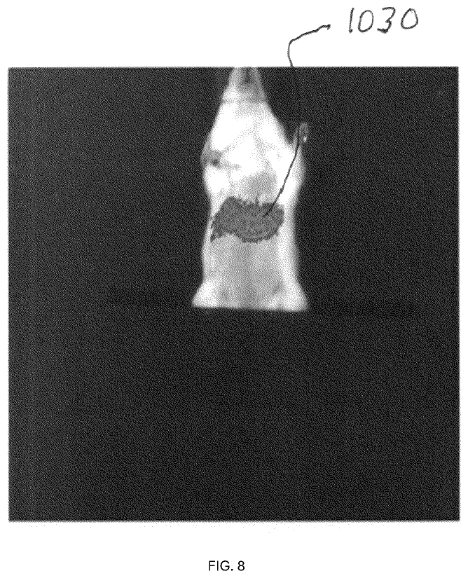

FIG. 8 shows in vivo luciferase transfection of mouse liver following intravenous (IV) treatment with FLuc-mRNA loaded PLNPs. The PLNPs were synthesized as set out above using the commercially available FLuc-mRNA. Balb/c mouse were treated IV at a dose of 0.5 mg/kg of mRNA and image at 6 hours post treatment using the xenogen microscope. Turning to FIG. 8, the transfected liver cells are labelled 1030 and are shown as having luminescence (which if viewable in color shows a substantially green region with a yellow and red interior region and a loose border region of blue). Colors green, yellow, red, and blue within the region of transfected liver cells 1030 are indicative of luminescence beyond the baseline level.

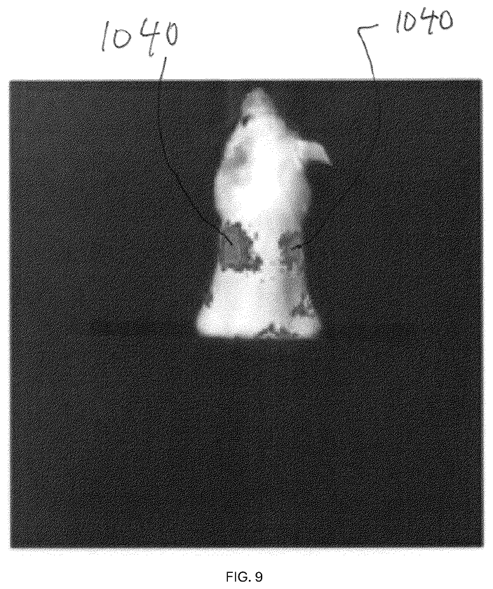

FIG. 9 shows in vivo luciferase transfection of mouse spleen and lymph nodes following intravenous (IV) treatment with FLuc-mRNA loaded PLNPs. The PLNPs were synthesized as described above using the commercially available FLuc-mRNA. Balb/c mouse were treated IV at a dose of 0.5 mg/kg of mRNA and imaged at 6 hours post treatment using the xenogen microscope. Turning to FIG. 9, the transfected spleen and lymph cells are labelled 1040 and are shown (if viewable in color) in green, which is indicative of luminescence beyond the baseline level.

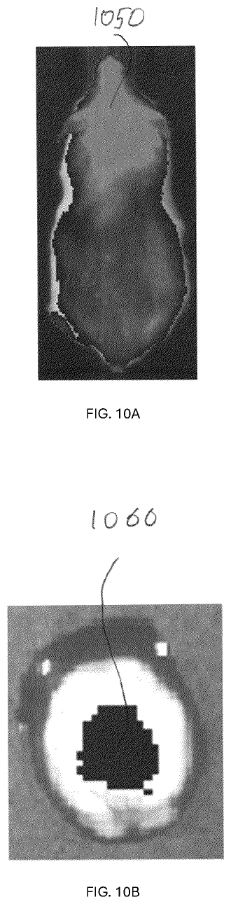

FIG. 10 shows in vivo brain delivery of mRNA following intravenous (IV) treatment with Cy5 labelled Fluc-mRNA loaded PLNPs. The PLNPs were synthesized as described above using the commercially available Cy5 labelled FLuc-mRNA. Balb/c mouse were treated IV at a dose of 1.5 mg/kg of mRNA and image at 6 hours post treatment using the xenogen microscope to show epi-fluorescence indicative of the mRNA signal in brain 1050 (which in color would appear as a solid yellow region surrounded by red; FIG. 10A). Ex vivo image of brain was also taken to confirm the mRNA delivery 1060 in the brain (which in color would appear as a solid brown inner region; FIG. 10B—note if viewing in color that the color thresholds are different for images FIG. 10).

### Additional Graft Co-Polymer Synthesis

As an additional Example of the breadth of potential anionic polymers usable in the PLNCs described herein, a further terpolymer was synthesized. A solution containing 2,2′-Azobis(2-methylpropionitrile) (AIBN), 2-Cyanoprop-2-yl-dithiobenzoate (CDB), MMA (PS80), and 6-acrylate D-glucose were dissolved into ethanol and purged with N₂ to remove any dissolved oxygen, then heated to 65° C. under magnetic stirring for overnight. After reaction, the resulting material was purified by precipitation into toluene and dried under dynamic vacuum for 24 h, then stored in a desiccator for future use.

The invention is not intended to be limited to the embodiments described herein, but rather the invention is intended to be applied widely within the scope of the inventive concept as defined in the specification as a whole including the appended claims.

1. A method of synthesizing charge switchable polymer-lipid nanoparticles (PLNPs) encapsulating a cargo, comprising the steps of:
solubilizing lipids in an alcohol or organic solvent;
solubilizing the cargo in an acidic aqueous solvent;
solubilizing an anionic polymer in an aqueous solvent;
rapidly mixing the solubilized lipids and cargo under stirring to obtain a lipid/cargo complex;
adding the solubilized anionic polymer aqueous solution to the lipid/cargo complex to form a polymer/lipid-cargo emulsion; and
homogenizing the polymer/lipid-cargo emulsion to obtain cargo loaded PLNPs, wherein the lipids are a mixture of ionizable cationic lipids, cholesterol, and helper lipids and wherein the anionic polymer is an amphiphilic polymer having a backbone and side chains, each of which includes carboxylic groups along at least a portion of its length.

2. The method of claim 1, wherein a stoichiometric ratio of protonatable nitrogen in the lipids to anionic phosphate in the nucleic acid cargo (N/P ratio) is in the range of 2:1 to 8:1, and the cargo is a nucleotide.

3. (canceled)

4. The method of claim 1, wherein the lipid mixture has a ratio of ionizable cationic lipids:cholesterol:helper lipids in the range of 30:30:40 to 70:20:10 or in the range of 40:40:20 to 60:30:10.

5. (canceled)

6. (canceled)

7. (canceled)

8. The method of claim 1, wherein the PNLPs are less than about 500 nm in diameter.

9. The method of claim 1 where the cargo is nucleotide-based.

10. (canceled)

11. The method of claim 8 where the cargo is selected from the group consisting of siRNA, mRNA, miRNA, RNA, RNAi, antisense oligonucleotides and DNA.

12. The method of claim 1 where a weight ratio of anionic polymer to lipids is between 0.01:1 to around 10:1.

13. (canceled)

14. (canceled)

15. (canceled)

16. The method of claim 1, wherein the anionic polymer is made from one or more of polysaccharide, chitosan, chitosan derivatives, polyacrylic acid, poly(methyl methacrylate/methacrylic acid), poly(butadiene/maleic acid), poly(ethylene-alt-maleic anhydride), or poly(meth acrylamide).

17. The method of claim 1, wherein the cationic ionizable lipids are ionizable fatty acid or phospholipid lipids with pKa below pH 7.0.

18. The method of claim 17, wherein the phospholipids are selected from the group consisting of the ionizable phospholipids Dlin-MC3-DMA, Lipid H (SM-102), Dlin-KC2-DMA, DlinDMA, YSK13-C3, L319, 304O13, CKK-E12, 503O13, C12-200, Lipid 10, ALC-0315, C14-4, and 306i10.

19. (canceled)

20. A charge switchable polymer-lipid nanoparticle (PLNP) comprising:
an anionic polymer, the anionic polymer being an amphiphilic polymer having a backbone and side chains, each of which includes carboxylic groups along at least a portion of its length;
ionizable cationic lipids;
cholesterol;
a helper lipids;
a cargo,
wherein the ionizable cationic lipids form a complex with the cargo,
wherein the anionic polymer forms a shell surrounding the complex, and
wherein the shell presents a negative charge under physiological conditions and protonates when the pH is below 6.

21. The PLNP of claim 20, wherein the PLNP has a N/P ratio in the range of 2:1 to 8:1 and the cargo is a nucleotide.

22. (canceled)

23. The PLNP of claim 20 where the ratio of ionizable cation lipids:cholesterol:helper lipids is in the range of 30:30:40 to 70:20:10 or in the range of 40:40:20 to 60:30:10.

24. (canceled)

25. (canceled)

26. (canceled)

27. The PLNP of claim 20, wherein the PNLPs are less than about 500 nm in diameter.

28. The PLNP of claim 20, wherein the cargo is nucleotide-based.

29. The PLNP of claim 27 where the cargo is selected from the group consisting of siRNA, mRNA, miRNA, tRNA, RNAi, antisense oligonucleotides and DNA.

30. The PLNP of claim 20, wherein a weight ratio of anionic polymer to lipids is between 1.01:1 to 10:1.

31. (canceled)

32. (canceled)

33. (canceled)

34. The PLNP of claim 20, wherein the anionic polymer is made from one or more of polysaccharide, chitosan, chitosan derivatives, polyacrylic acid, poly(methyl methacrylate/methacrylic acid), and poly(butadiene/maleic acid), poly(ethylene-alt-maleic anhydride), or poly(meth acrylamide).

35. The PLNP of claim 20, wherein the cationic ionizable lipids are ionizable fatty acid or phospholipids lipids with pKa below pH 7.0.

36. The PLNP of claim 20 where the phospholipids are selected from the group consisting of the ionizable phospholipids Dlin-MC3-DMA, Lipid H (SM-102), Dlin-KC2-DMA, DlinDMA, YSK13-C3, L319, 304O13, CKK-E12, 503O13, C12-200, Lipid 10, ALC-0315, C14-4, and 306i10.

37. (canceled)

38. (canceled)

39. (canceled)

40. (canceled)
